(19) United States
(12) Patent Application Publication
(10) Pub. No.: US 2023/0061704 A1
(43) Pub. Date: Mar. 2, 2023

(54) SYSTEM AND METHOD FOR DETERMINATION OF DRY WEIGHT BY MULTI-FREQUENCY BIOIMPEDANCE MEASUREMENTS

(71) Applicant: SeeMedX, Inc, Shingle Springs, CA (US)

(72) Inventors: Philip Hamski, Shingle Springs, CA (US); Robert Rinehart, Shingle Springs, CA (US); Dios Aba, Shingle Springs, CA (US); Marc O. Griofa, Shingle Springs, CA (US)

(73) Assignee: SeeMedX, Inc, Shingle Springs, CA (US)

(21) Appl. No.: 17/899,984

(22) Filed: Aug. 31, 2022

Related U.S. Application Data
(60) Provisional application No. 63/239,082, filed on Aug. 31, 2021.

(51) Int. Cl. A61B 5/0537; A61B 5/00

(52) U.S. Cl. CPC A61B 5/0537; A61B 5/4869; A61B 5/4875

(57) ABSTRACT

A dry weight measurement system and method uses multiple frequency bioimpedance measurements. In one embodiment, the dry weight measurement system may be used to monitor dialysis patients.

### RELATED APPLICATIONS

This application claims the benefit under 35 USC 119(e) to U.S. Provisional Application No. 63/239,082, filed Aug. 31, 2021 and which is incorporated herein by reference.

### FIELD

The disclosure relates to a device using multiple frequencies bioimpedance measurements to measure dry weight and in particular to a fluid monitor that uses multiple frequencies bioimpedance measurements to measure dry weight.

### BACKGROUND

Accurate assessment of hydration status and specification of dry weight (DW) are major problems in the clinical treatment of hemodialysis (HD) patients. Bioelectrical impedance analysis (BIA) [bioimpedance] has been recognized as a noninvasive and simple technique for the determination of DW in HD patients. DW may be defined as the target post-HD weight at which the patient is as close as possible to a normal hydration state without experiencing symptoms that are indicative of over- or underhydration at or after the end of HD treatment. In the clinical practice of HD, postdialysis DW is estimated by trial and error, and the degree of imprecision is reflected in the development of intradialytic symptoms or chronic volume overload with poor control of blood pressure (BP). Traditionally, the presence of a proper amount of blood in the body (known as euvolemia) in dialysis patients is achieved by the application of clinical criteria such as absence of symptomatic dialysis associated hypotension, by arterial normotension in the dialysis interval without the need for antihypertensive medications, or by absence of any signs or symptoms of hypotension or hypertension. Thus, it is desirable to be able to measure dry weight using multiple frequency bioimpedance measurements and it is to this end that the disclosure is directed.

### BRIEF DESCRIPTION OF THE DRAWINGS

FIG. 1 is a front perspective view of the front face of a base unit of a fluid monitor;

FIG. 2 is a block diagram of the major circuit components of the base unit of the impedance monitor;

FIG. 3 illustrates a method to measure calf resistivity using a fluid monitor;

FIG. 4 shows an example of the Ro/Rt curve during ultrafiltration;

FIG. 5 shows a determination of dry weight using calf resistivity and bioimpedance measurements;

FIG. 6 shows raw filtered data from the fluid monitor;

FIGS. 7 and 8 shows the raw and filtered data at 100 and 5 kHz;

FIG. 9 shows the data that has been processed;

FIG. 10 is a graph showing the processed data;

FIG. 11 is a graph in which the 5 kHz and 100 kHz outputs are combined;

FIG. 12 shows the resultant data used to determining a flattening of the combined lines; and

FIG. 13 is chart showing the detection of Euvolemia with the flattened lines shown by the horizontal line.

### DETAILED DESCRIPTION OF ONE OR MORE EMBODIMENTS

The disclosure is particularly applicable to measuring dry weight for hemodialysis (HD) patients using multiple frequency bioimpedance measurements and it is in this context that the disclosure will be described. It will be appreciated, however, that the device, system and method has greater utility since the device and dry weight measurement technique using multiple frequency bioimpedance measurements can be used for other medical treatments or issues in which the dry weight issue arises.

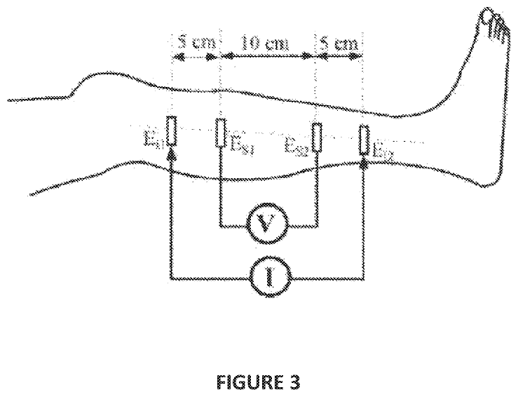

The dry weight of a patent can be measured using the fluid monitor and a patient interface that includes a four electrode array assembly which is coupled to a small, hand transportable base unit 20. The electrode assembly 100 may be placed on a body member, such as a leg as shown in FIG. 3. The electrode assembly 100 may have a set of four electrodes (for example E₁₁, E_{S1}, E_{S2} and E₁₂ as shown in FIG. 3) that are axially aligned. In one embodiment, a excitation signal (at multiple frequencies) is delivered to the patient through the first and fourth electrodes (E₁₁ and E₁₂) while the signals from the patient's body are received at the second and third electrodes (E_{S1}, E_{S2}) and used to determine the dry weight or euvolumia of the patient. In one embodiment, these determinations are made during and after a HD treatment.

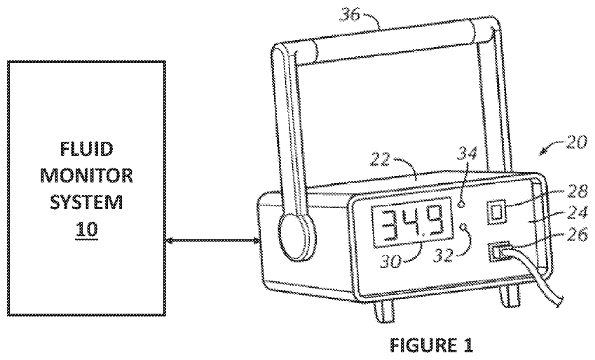

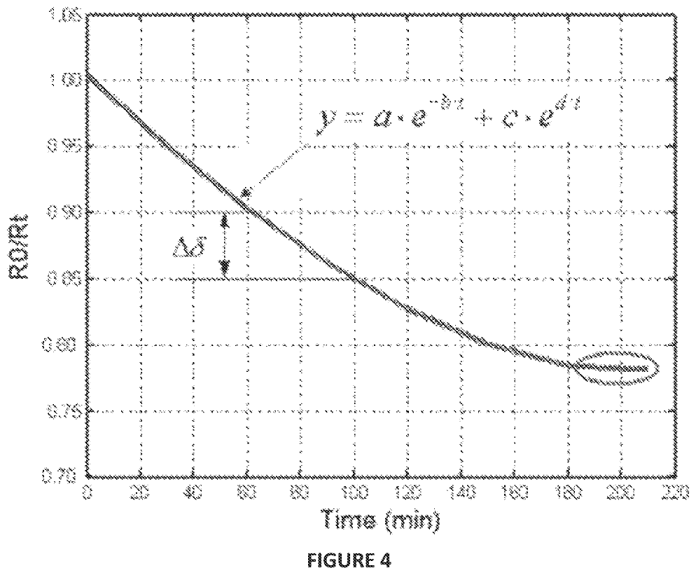

FIG. 1 depicts a front panel 24 of the base unit 20. The base unit 20 is relatively lightweight and is contained in a relatively small (i.e. hand transportable) housing 22. The base unit 20 may be electrically and communicately coupled to a fluid monitor system 10 that may be implemented as a computer system that has at least one processor. Preferably, the base unit 20 is provided with a handle 36 to facilitate transport. The base unit 20 contains several user interfaces in addition to a connector port 26 for receiving a connection end of the electrode array assembly 100. These interfaces preferably include a display 30 (e.g. formed by three, seven segment LED's) which preferably digitally display impedance as xx.x ohms, a start switch 28 to start the system 10, a low battery alert 32, and a cable disconnect alert 34. Preferably the base unit 20 also contains a beeper 86 (see FIG. 4) or other sound generator for signaling purposes. Alternatively, additional alert lights (not illustrated) could be substituted for the beeper 86. Alternatively, the display 30 may be able to display the different types of data shown in FIG. 4.

The base unit 20 is preferably configured to perform all necessary steps to measure, determine and display the patient's base impedance after the start switch 28 is actuated. However, the system 10 does not provide any patient diagnostic parameters. That is, it provides only a measurement of impedance over a predetermined fixed length of the patient's body. This value can be compared with other impedance values for the patient or against limit values and used as a relative measure of patient's “dryness” or “level of hydration”. An analogy will be a blood pressure instrument which displays patient's systolic and diastolic blood pressure, but does not diagnose if a patient has hypertension or not. The information provided by system 10 will be evaluated along with various other parameters by health care or other professional to identify the use of the information for their specific purpose.

The base unit 20 will provide the following outputs. The LED display 30 preferably will display impedance value as xx.x. During measurement, a rotating/flickering pattern can be displayed to indicate the measurement is in progress. To ensure that the user U records ONLY the impedance values, the system software preferably will not display any numerical values other than impedance value. This means that there should be no countdown timers and no error or diagnostic codes expressed as numerical values. The base unit 20 will also indicate an error condition in the event it detects that it could not perform a valid impedance measurement or that the impedance value was outside of a predetermined measurement range (suggestedly 5 to 55 ohms). If the electrode array assembly is disconnected from the system cable disconnect alert 34 will alarm.

The base unit 20 may activate the low battery indication light 32 in the event it detects that the battery voltage is below a level that will allow for reliable impedance measurement. In the event of a low battery voltage condition, the base unit 20 may blink this LED 32, for example at a rate of once every 10 (+/−0.5) seconds for a period of 30 (+/−2) sec. If the battery voltage drops below 5.25 volts, but remains above 4.75 volts, the impedance results will be displayed along with blinking-battery condition LED 32 to indicate that the battery power is getting low but still acceptable. If the battery voltage drops below 4.75 volts, both LEDs 32, 34 can be made to blink to indicate that the battery voltage is low and accurate results could not be displayed. Preferably a micro-controller 80 in the base unit 20 will continue to operate below 4.75 volts, even though an accurate measurement cannot be made, to warn the user of the condition of the unit.

The base unit 20 can be configured to provide various beeper alerts to the user. Preferably the base unit 20 beeps to indicate that the measurement is completed and the displayed value should be recorded. The beeper 86 may further be activated to indicate other, different conditions or steps, for example, when the base unit 20 is initially activated, while the unit is initializing, while the power supply is stabilizing, while measurements are being taken and/or before the unit shuts itself off. The beeper 86 can also be activated in the event a successful measurement was not accomplished or an error condition was detected. Different beep patterns are suggestedly used for different conditions including different states of the base unit 20.

FIG. 3 illustrates an example of the hardware and circuitry 40 of the base unit 20 that may include signal generating circuitry 50, voltage detection circuitry 60 and impedance calculation circuitry 70. The impedance calculation circuitry 70 includes an analog/digital converter 72, data acquisition circuitry 74, and data analysis and storage circuitry 76. Along with power management circuitry 82, the impedance calculation circuitry 70 is provided by a micro-controller 80.

The signal generating circuitry 50 generates the stable excitation current (I). A current source subcircuit 52 includes a constant current source circuit to supply a current of about 1 mA or less, preferably a 0.98+−0.01 mA, at a 100+/−1% and 5+/−1% kHz frequency to the first and fourth electrodes through a amplifier and filter circuit, the connection cord 130 and electrode array lead 110. The current source subcircuit 52 is configured to output a current of less than 1 mA under all conditions including equipment component failure. The wave form of the current is suggestedly sinusoidal with less than ten percent total harmonic distortion. Voltage values across two of the four electrodes, preferably the second and third electrodes are passed to an-RMS to DC converter 62 then to an amplifier-to a low pass filter subcircuit 64. The low pass filter subcircuit 64 functions to remove extraneous electrical interference from ambient sources, for example, home appliances operating on standard residential 60 Hz current. A preferred cut-off frequency of the low pass filter subcircuit 64 is about 50 Hz. The base unit 20 measures voltage developed across detection electrodes 122, 124 when the excitation current source is energized.

Micro-controller 80, which might be a PIC 16F873 device, controls generation of the excitation current and receives the filtered voltage analog signal from the amplifier and low pass filter 64 at the input of analog to digital converter 72. Suggestedly the injected current is not generated for a short period of time (e.g. fifteen to thirty seconds) after the start switch 28 is actuated to allow the user to settle into a quiescent state. Suggestedly the current is then injected for a predetermined period, e.g. thirty seconds, to perform the measurement. Voltage values sampled from the A/D converter 72 are received by the data acquisition circuitry 74 of the micro-controller 80 suggested at a rate of about five samples per second for all or most of the thirty second period. Data analysis and storage circuitry 76 of micro-controller 80 sums the counts generated by the A/D converter 72, divides sum by the total number of samples taken to provide an average voltage value which is converted into an impedance value. The algorithm used for generating impedance in tenths of ohms is: averaged A/D counts*Gain+Offset, where in the preferred circuit the Gain is 0.6112 and the Offset is 1.1074. Gain and Offset are based on the electronics design and operating range and are used for all base units 20. Each system 10 is calibrated to match the use of these numbers. The data analysis circuitry 76 also controls the various displays 30, 32, and 34. The power management circuitry 82 controls the generation and distribution of power in the base unit circuitry 40 to control operation of the system 10. Specific functions of the power management circuitry 82 include a first function 82a of providing power to the processor; a second function 82b of providing power to the AID converter, and a third function 82c of monitoring the input voltage. A power supply 90 may be provided by conventional dry-cell batteries (not shown) or by an external power adapter (not shown) connected to a conventional 120 V outlet.

The base unit 20 may be provided with a serial port 84 to work with logic level and USB signals. The timing for the serial data can be similar to RS232 signal or other conventional data transfer format. The base unit 20 would preferably be provided with a serial port, for example one configured to operate at 9600 baud, with 8 bit data, 1 Start bit, 1 Stop bit and no parity bit format. An external level translator may be necessary to interface the base unit to a PC or a handheld or portable device. Upon receipt of a specific command, the base 20 unit would be configured to transmit the information related to all or a subset (e.g. the last ten) of the readings of the impedance measurement. This information may also include the date and time of measurement, impedance value, and/or the serial number of the unit.

Bioelectrical impedance analysis (BIA) [bioimpedance] measures the impedance or opposition to the flow of an electric current through the body fluids contained mainly in the lean and fat tissue. Impedance is low in lean tissue, where intracellular fluid and electrolytes are primarily contained, but high in fat tissue. Impedance is thus proportional to body water volume (TBW). The impedance of a biological tissue comprises two components, the resistance and the reactance. The conductive characteristics of body fluids provide the resistive component, whereas the cell membranes, acting as imperfect capacitors, contribute a frequency-dependent reactive component.

In different embodiments the microcontroller 80 of the device 20 or a processor of the fluid monitor system 10 may store and execute a plurality of lines of instructions/computer code that are executed by the processor/microcontroller so that the processor/microcontroller is configured to perform the dry weight, hydration, impedance or euvolemia determinations/methods discussed in more detail below. The determination/method may used to measured hydration or impedance to perform the determinations for the patient to which the unit 20 is connected by the adhered electrodes.

### First Embodiment of Calf Resistivity Measurements

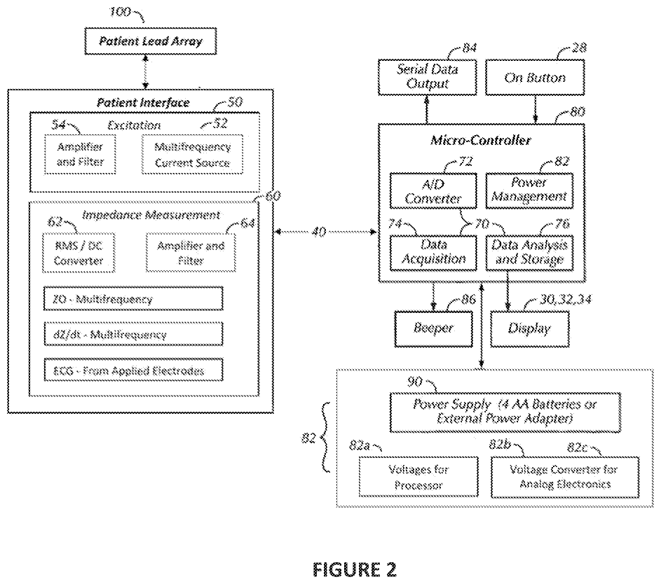

The resistivity (ps, p100) of a calf portion of a user is calculated from resistance [impedance] (Rs, R100) at 5 kHz and 100 kHz using the Fluid Status Monitor system 10 and device 20 shown in FIGS. 1-2 where A and L are the cross-sectional areas of the calf and the distance (10 cm) between the sensing electrodes, respectively. FIG. 3 shows a set-up for the calf resistivity measurement using the fluid monitor. As shown in FIG. 3, in one implementation, two electrodes may be placed on the outer side of calf as shown in FIG. 3 to inject an alternating current (0.8 mA/5 kHz; 2.0 mA/100 kHz) and measure voltage using the Fluid Status Monitor 10, 20 (not shown in FIG. 3). One sensing electrode (Es1) is placed on the point of maximal calf circumference (CMax); while the other sensing electrode (Es2) is placed 10 cm below the Es1 which is defined at the point as minimal circumference (CMin).

Thus, for the measurements, the cross sectional area of the calf is calculated where Cave is the mean of the two measured calf circumferences (Cave=(CMax+Cmin)/2). In addition, A=Cave2/(4n). To reduce the effect of differences in body composition, resistivity ps is normalized by the body mass index (BMI; calculated as body mass BM [kg] divided by height2 [m]2), and reported as calf normalized resistivity (PN,$) in units of Qm3/kg.

### Continuous Calf Bioimpedance Measurement

The calf bioimpedance at 5 kHz and 100 kHz using the Fluid Status Monitor system and device 10, 20 (in which the monitor generates these two different frequency signals) provides a way to continuously measure calf extracellular (ECV) and intracellular (ICV) volume during hemodialysis (HD). Relative change in ECV can be represented by Ro/Rt where REo and REt are extracellular resistance [impedance] at the beginning and at any time until the end of HD. Since the plasma volume only takes about ten percent of ECV in the calf, changes in Reo/REt mainly indicate change in interstitial fluid volume.

In previous studies, when the curve of REolREt was flattening, it indicated a limitation of excess fluid in interstitial space so that the state of this hydration can be defined as the normal hydration state. FIG. 4 shows the principle of the continuous monitoring Ro/Rt until reaching the flattening portion (as indicated by the oval) in last 20 minutes of treatment. In FIG. 4, the curve can be represented as exponential function of two variables and two constants (a and c).

The main advantage with curve of Ro/Rt is the method does not require a normal range from a healthy population but utilizes its slope change as an indicator of tissue hydration. Flattening of the curve at both 5 kHz and 100 kHz during the final 20 minutes of treatment has been defined as excess fluid volume being completely removed in the body when the patient has reached euvolemia or dry weight.

### Determination of Dry Weight Using Calf Resistivity and Bioimpedance Measurements.

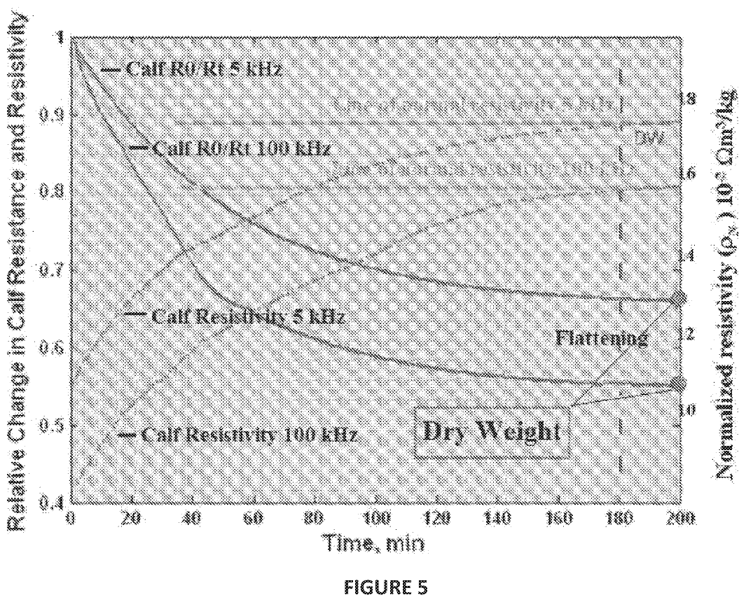

The principle of using Ro/Rt curve at 5 kHz/1OO kHz and normalized resistivity is shown in FIG. 5 that shows a determination of dry weight using calf resistivity and bioimpedance measurements. The slope of change in resistance [impedance] represents the removal of excess fluid volume in the calf of the patient. The flattening of the resistance [impedance] curve at both 5 kHz and I 00 kHz means that the fluid exchange between intravascular and interstitial compartments has reached an equilibrium state. Euvolemia and dry weight has been reached if the curve is flattening and the normalized resistivity is in the normal range for both 5 kHz and I 00 kHz as shown in FIG. 5.

Approaching normalized resistivity value in the calf provides a secondary indication of euvolemia and dry weight. The Fluid Status Monitor 10, 20 measures extracellular resistance [impedance] (5 kHz) and whole segment resistance [impedance] (100 kHz). The delta of these two measurements provides an indication of intracellular resistance [impedance]. The algorithm used to determine normal hydration state employs two criteria together: a) flattening of the change in resistance [impedance] (Ro/Rt) curve at both 5 kHz and 100 kHz; and b) normalized resistivity in the range derived from healthy subjects at both 5 kHz and I 00 kHz. The combination of these elements provides four distinct criteria that can be cross-referenced to identify when the patient has reached post-dialysis dry weight or a euvolemic state that is an objective repeatable method.

### Second Embodiment of Calf Resistivity Measurements and Determination of Dry Weight by Calf Resistivity and Bioimpedance Measurements

As above, calf resistivity (ρ₅, ρ₁₀₀) may be calculated from resistance [impedance] (R₅, R₁₀₀) at 5 kHz and 100 kHz and where A and L are the cross-sectional areas of the calf and the distance (10 cm) between the sensing electrodes, respectively.

$${ρ}_{5} = {R}_{5} X \frac{A}{L} ( Ω \cdot cm ) \qquad ( 1 a )$$

$${ρ}_{100} = {R}_{100} X \frac{A}{L} ( Ω \cdot cm ) \qquad ( 1 b )$$

The cross sectional area is calculated as:

A=Cₐᵥₑ²/(4π) (2)

where Cₐᵥₑ is the mean of the two measured calf circumferences (Cₐᵥₑ=(C_{Max}Cₘᵢₙ)/2).

Similar to the above embodiment, to reduce the effect of differences in body composition, resistivity ρ₅ is normalized by the body mass index (BMI; calculated as body mass BM [kg] divided by height² [m]²), and reported as calf normalized resistivity (ρ_{N,5}) in units of Ωm³/kg (Equations 3a and 3b).

$${ρ}_{N , 5} = \frac{{ρ}_{5}}{BMI} \qquad ( 3 a )$$

$${ρ}_{100} = \frac{{ρ}_{100}}{BMI} \qquad ( 3 b )$$

### Continuous CalfBioimpedance Measurement

Calfbioimpedance at 5 kHz and 100 kHz using the Fluid Status Monitor 10, 20 provides a way to continuously measure calf extracellular (ECV) and intracellular (ICV) volume during hemodialysis (HD). Relative change in ECV can be represented by Ro/Rt where R_{Eo} and R_{Et} are extracellular resistance [impedance] at the beginning and at any time until the end of HD. Since the plasma volume only takes about ten percent of ECV in the calf, changes in R_{Eo}/R_{Et} mainly indicate change in interstitial fluid volume. The ratio of R_{Eo}/R_{Et} indicates the ratio of extracellular fluid volume in any time (ECVₜ) to the volume at the beginning (ECV₀). The relationship can be described as following (Zhu et al., 2004):

$$\frac{E C {V}_{t}}{E C {V}_{0}} = \frac{{ρ}_{ECV} \frac{{L}^{2}}{{R}_{E t}}}{{ρ}_{ECV} \frac{{L}^{2}}{{R}_{E 0}}} = \frac{\frac{1}{{R}_{E t}}}{\frac{1}{{R}_{E 0}}} = \frac{{R}_{E 0}}{{R}_{E t}} \qquad ( 4 )$$

where ρ_{ECV} is the resistivity in extracellular fluid volume, L is the length of the measurement area of the calf and R_{E0} and R_{Et} are extracellular resistance at beginning and at any time during dialysis respectively. Since ρ_{ECV} and L are constant in equation 4, they are canceled in the ratio of volume so that R_{Eo}/R_{Et} is equal to ECVₜ/ECV₀. This is the principle why decrease in calf R_{Eo}/R_{Et} represents the reduction of extracellular fluid volume. In previous studies, when the curve of R_{Eo}/R_{Et} was flattening, it indicated a limitation of excess fluid in interstitial space so that the state of this hydration can be defined as normal hydration state. Like the above embodiment, FIG. 4 shows the principle of the continuous monitoring R_{Eo}/R_{Et} until reaching the flattening portion (as shown by the oval) in last 20 minutes of treatment.

Like the first embodiment, the main advantage with curve of R_{Eo}/R_{Et} is the method does not require a normal range from a healthy population but utilizes its slope change as an indicator of tissue hydration. Flattening of the curve at both 5 kHz and 100 kHz during the final 20 minutes of treatment has been defined as excess fluid volume being completely removed in the body when the patient has reached euvolemia or dry weight.

### Continuous Measurement of Resistivity

To continuously measure calf resistivity, calf circumference must be measured because at the same time the cross-sectional area is reducing during the treatment. The major issue of continuously calculating resistivity is how to measure the calf circumference during HD. The calf cross sectional area during HD can be calculated based on an assumption that change in circumference during dialysis is due to decrease in the fluid volume of the calf Therefore, the value of circumference can be calculated as:

$${χ}_{t} = \sqrt{{χ}_{0}^{2} - \frac{4 π {ρ}_{0} L}{{R}_{E , 0}} ( 1 - \frac{{R}_{E , 0}}{{R}_{E , t}} )} \qquad ( 5 )$$

where χ₀ and R_{E,0} are measured at the start of dialysis, ρ₀ is a resistivity with constant value which is experimentally calibrated by actual measurements of circumference, L is 10 cm, R_{E0} and R_{Et} are resistance [impedance] measured by the Fluid Status Monitor 10, 20 initially and continuously measured until the end of the treatment. Using equation 5 above, calf circumference can be accurately calculated (Zhu et al., 2006b).

### Determination of Dry Weight Using Calf Resistivity and Bioimpedance Measurements

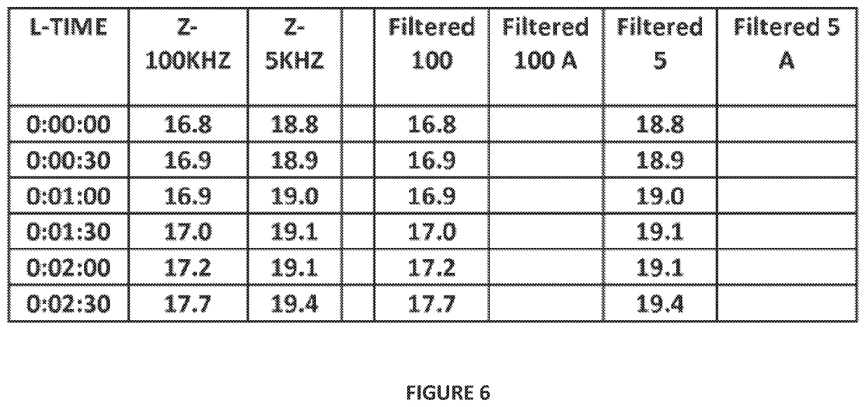

The principle of using R_{Eo}/R_{Et} curve at 5 kHz/100 kHz and normalized resistivity is shown, as with the first embodiment, in FIG. 6. The slope of change in resistance [impedance] represents the removal of excess fluid volume in the calf The flattening of the resistance [impedance] curve at both 5 kHz and 100 kHz means that the fluid exchange between intravascular and interstitial compartments has reached an equilibrium state. Euvolemia and dry weight has been reached if the curve is flattening and the normalized resistivity is in the normal range for both 5 kHz and 100 kHz as shown in FIG. 5.

### Embodiment of Calculation of Euvolemia

FIG. 6 shows raw filtered data from the fluid monitor;

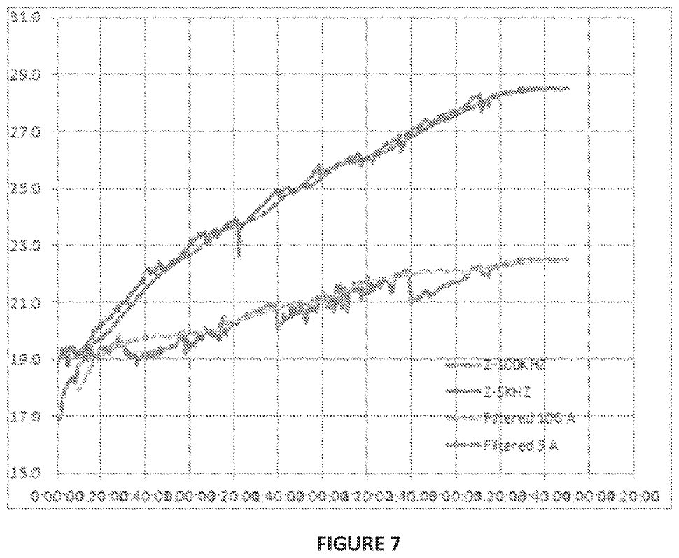

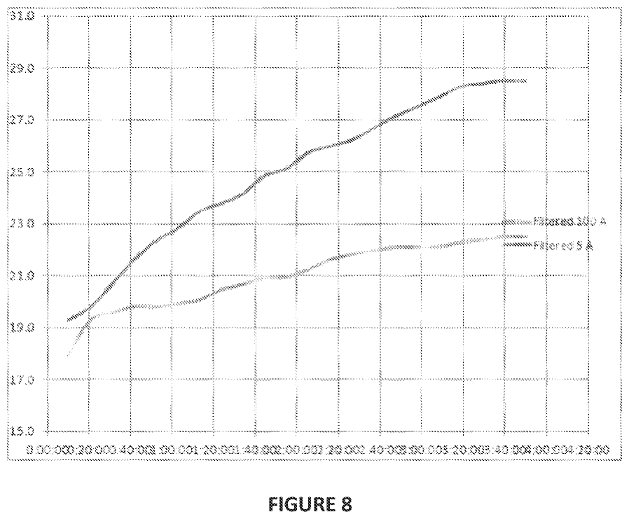

FIGS. 7 and 8 shows the raw and filtered data at 100 and 5 kHz;

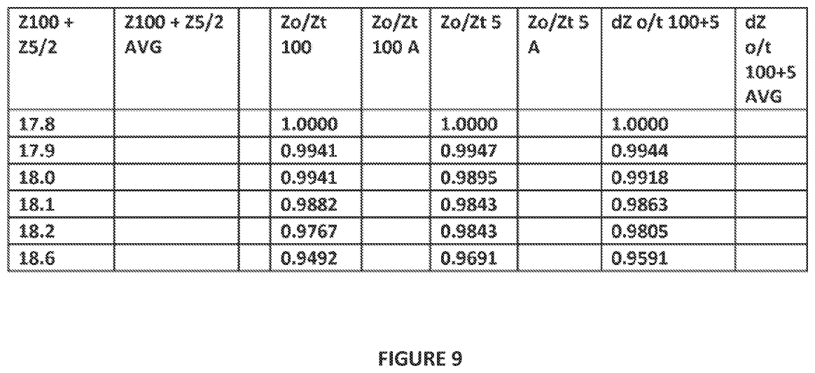

FIG. 9 shows the data that has been processed;

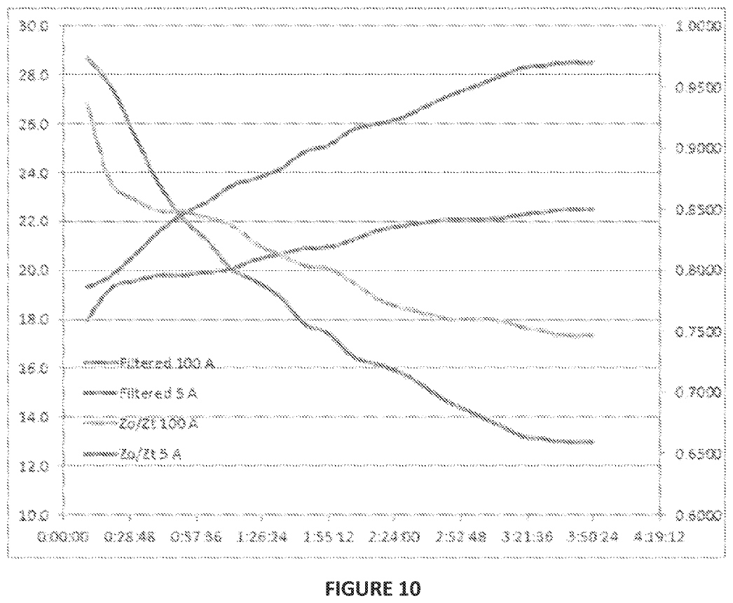

FIG. 10 is a graph showing the processed data;

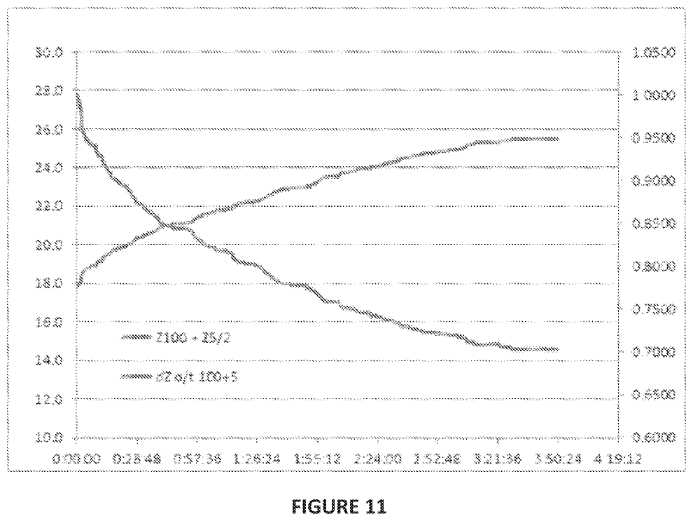

FIG. 11 is a graph in which the 5 kHz and 100 kHz outputs are combined;

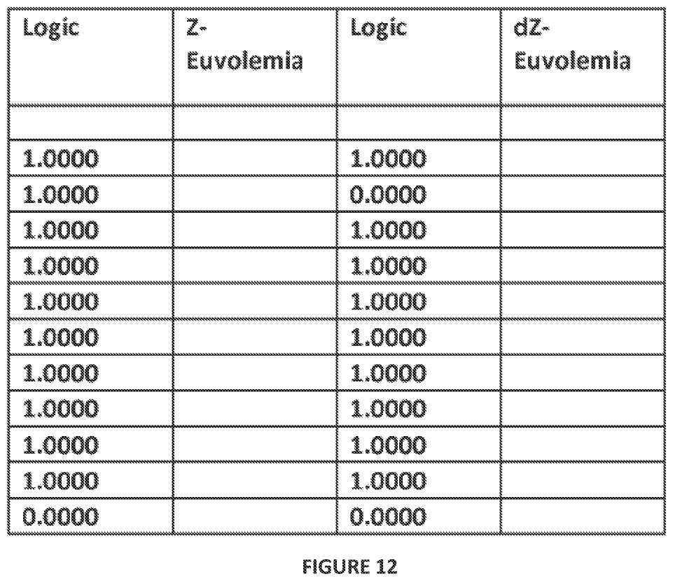

FIG. 12 shows the resultant data used to determining a flattening of the combined lines; and

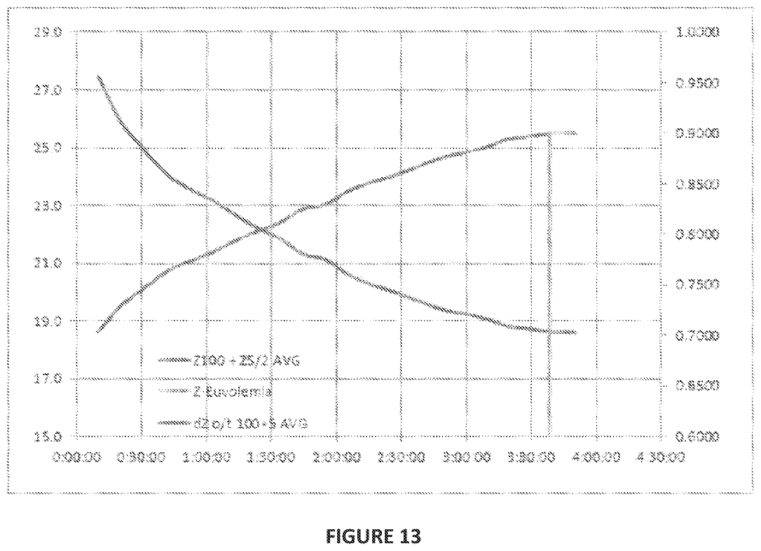

FIG. 13 is chart showing the detection of Euvolemia with the flattened lines shown by the horizontal line.

The foregoing description, for purpose of explanation, has been with reference to specific embodiments. However, the illustrative discussions above are not intended to be exhaustive or to limit the disclosure to the precise forms disclosed. Many modifications and variations are possible in view of the above teachings. The embodiments were chosen and described in order to best explain the principles of the disclosure and its practical applications, to thereby enable others skilled in the art to best utilize the disclosure and various embodiments with various modifications as are suited to the particular use contemplated.

The system and method disclosed herein may be implemented via one or more components, systems, servers, appliances, other subcomponents, or distributed between such elements. When implemented as a system, such systems may include and/or involve, inter alia, components such as software modules, general-purpose CPU, RAM, etc. found in general-purpose computers. In implementations where the innovations reside on a server, such a server may include or involve components such as CPU, RAM, etc., such as those found in general-purpose computers.

Additionally, the system and method herein may be achieved via implementations with disparate or entirely different software, hardware and/or firmware components, beyond that set forth above. With regard to such other components (e.g., software, processing components, etc.) and/or computer-readable media associated with or embodying the present inventions, for example, aspects of the innovations herein may be implemented consistent with numerous general purpose or special purpose computing systems or configurations. Various exemplary computing systems, environments, and/or configurations that may be suitable for use with the innovations herein may include, but are not limited to: software or other components within or embodied on personal computers, servers or server computing devices such as routing/connectivity components, hand-held or laptop devices, multiprocessor systems, microprocessor-based systems, set top boxes, consumer electronic devices, network PCs, other existing computer platforms, distributed computing environments that include one or more of the above systems or devices, etc.

In some instances, aspects of the system and method may be achieved via or performed by logic and/or logic instructions including program modules, executed in association with such components or circuitry, for example. In general, program modules may include routines, programs, objects, components, data structures, etc. that perform particular tasks or implement particular instructions herein. The inventions may also be practiced in the context of distributed software, computer, or circuit settings where circuitry is connected via communication buses, circuitry or links. In distributed settings, control/instructions may occur from both local and remote computer storage media including memory storage devices.

The software, circuitry and components herein may also include and/or utilize one or more type of computer readable media. Computer readable media can be any available media that is resident on, associable with, or can be accessed by such circuits and/or computing components. By way of example, and not limitation, computer readable media may comprise computer storage media and communication media. Computer storage media includes volatile and nonvolatile, removable and non-removable media implemented in any method or technology for storage of information such as computer readable instructions, data structures, program modules or other data. Computer storage media includes, but is not limited to, RAM, ROM, EEPROM, flash memory or other memory technology, CD-ROM, digital versatile disks (DVD) or other optical storage, magnetic tape, magnetic disk storage or other magnetic storage devices, or any other medium which can be used to store the desired information and can accessed by computing component. Communication media may comprise computer readable instructions, data structures, program modules and/or other components. Further, communication media may include wired media such as a wired network or direct-wired connection, however no media of any such type herein includes transitory media. Combinations of the any of the above are also included within the scope of computer readable media.

In the present description, the terms component, module, device, etc. may refer to any type of logical or functional software elements, circuits, blocks and/or processes that may be implemented in a variety of ways. For example, the functions of various circuits and/or blocks can be combined with one another into any other number of modules. Each module may even be implemented as a software program stored on a tangible memory (e.g., random access memory, read only memory, CD-ROM memory, hard disk drive, etc.) to be read by a central processing unit to implement the functions of the innovations herein. Or, the modules can comprise programming instructions transmitted to a general-purpose computer or to processing/graphics hardware via a transmission carrier wave. Also, the modules can be implemented as hardware logic circuitry implementing the functions encompassed by the innovations herein. Finally, the modules can be implemented using special purpose instructions (SIMD instructions), field programmable logic arrays or any mix thereof which provides the desired level performance and cost.

As disclosed herein, features consistent with the disclosure may be implemented via computer-hardware, software, and/or firmware. For example, the systems and methods disclosed herein may be embodied in various forms including, for example, a data processor, such as a computer that also includes a database, digital electronic circuitry, firmware, software, or in combinations of them. Further, while some of the disclosed implementations describe specific hardware components, systems and methods consistent with the innovations herein may be implemented with any combination of hardware, software and/or firmware. Moreover, the above-noted features and other aspects and principles of the innovations herein may be implemented in various environments. Such environments and related applications may be specially constructed for performing the various routines, processes and/or operations according to the invention or they may include a general-purpose computer or computing platform selectively activated or reconfigured by code to provide the necessary functionality. The processes disclosed herein are not inherently related to any particular computer, network, architecture, environment, or other apparatus, and may be implemented by a suitable combination of hardware, software, and/or firmware. For example, various general-purpose machines may be used with programs written in accordance with teachings of the invention, or it may be more convenient to construct a specialized apparatus or system to perform the required methods and techniques.

Aspects of the method and system described herein, such as the logic, may also be implemented as functionality programmed into any of a variety of circuitry, including programmable logic devices (“PLDs”), such as field programmable gate arrays (“FPGAs”), programmable array logic (“PAL”) devices, electrically programmable logic and memory devices and standard cell-based devices, as well as application specific integrated circuits. Some other possibilities for implementing aspects include: memory devices, microcontrollers with memory (such as EEPROM), embedded microprocessors, firmware, software, etc. Furthermore, aspects may be embodied in microprocessors having software-based circuit emulation, discrete logic (sequential and combinatorial), custom devices, fuzzy (neural) logic, quantum devices, and hybrids of any of the above device types. The underlying device technologies may be provided in a variety of component types, e.g., metal-oxide semiconductor field-effect transistor (“MOSFET”) technologies like complementary metal-oxide semiconductor (“CMOS”), bipolar technologies like emitter-coupled logic (“ECL”), polymer technologies (e.g., silicon-conjugated polymer and metal-conjugated polymer-metal structures), mixed analog and digital, and so on.

It should also be noted that the various logic and/or functions disclosed herein may be enabled using any number of combinations of hardware, firmware, and/or as data and/or instructions embodied in various machine-readable or computer-readable media, in terms of their behavioral, register transfer, logic component, and/or other characteristics. Computer-readable media in which such formatted data and/or instructions may be embodied include, but are not limited to, non-volatile storage media in various forms (e.g., optical, magnetic or semiconductor storage media) though again does not include transitory media. Unless the context clearly requires otherwise, throughout the description, the words “comprise,” “comprising,” and the like are to be construed in an inclusive sense as opposed to an exclusive or exhaustive sense; that is to say, in a sense of “including, but not limited to.” Words using the singular or plural number also include the plural or singular number respectively. Additionally, the words “herein,” “hereunder,” “above,” “below,” and words of similar import refer to this application as a whole and not to any particular portions of this application. When the word “or” is used in reference to a list of two or more items, that word covers all of the following interpretations of the word: any of the items in the list, all of the items in the list and any combination of the items in the list.

Although certain presently preferred implementations of the invention have been specifically described herein, it will be apparent to those skilled in the art to which the invention pertains that variations and modifications of the various implementations shown and described herein may be made without departing from the spirit and scope of the invention. Accordingly, it is intended that the invention be limited only to the extent required by the applicable rules of law.

While the foregoing has been with reference to a particular embodiment of the disclosure, it will be appreciated by those skilled in the art that changes in this embodiment may be made without departing from the principles and spirit of the disclosure, the scope of which is defined by the appended claims.

What is claimed is:

1. A method for assessing hydration for a patient using multiple frequency bioimpedance measurements, the method comprising:
generating a first frequency that measures an extracellular impedance of the patient during a treatment;
generating a second, higher frequency that measures an intracellular impedance of the patient during the treatment at the same time that the extracellular impedance is measured; and
determining, a dry weight of the patient during the treatment based on the extracellular impedance during the treatment.

2. The method of claim 1, wherein determining the dry weight of the patient further comprises determining a ratio of the extracellular impedance at a start of the treatment to the extracellular impedance at a predetermined later time during the treatment and determining the dry weight based on the determined ratio.

3. The method of claim 1, wherein the first frequency is 5 kHz and the second higher frequency is 100 kHz.

4. The method of claim 1 further comprising adhering an electrode array to the patient to measure the extracellular impedance and the intracellular impedance.

5. The method of claim 1, wherein the treatment is dialysis.

6. A method for assessing hydration for a patient using multiple frequency bioimpedance measurements, the method comprising:
generating a first frequency that measures an extracellular impedance of the patient during a treatment;
generating a second, higher frequency that measures an intracellular impedance of the patient during the treatment at the same time that the extracellular impedance is measured; and
determining, euvolemia of the patient during the treatment based on the extracellular impedance during the treatment.

7. The method of claim 6, wherein determining euvolemia of the patient further comprises determining a ratio of the extracellular impedance at a start of the treatment to the extracellular impedance at a predetermined later time during the treatment and determining the dry weight based on the determined ratio.

8. The method of claim 6, wherein the first frequency is 5 kHz and the second higher frequency is 100 kHz.

9. The method of claim 6 further comprising adhering an electrode array to the patient to measure the extracellular impedance and the intracellular impedance.

10. The method of claim 6, wherein the treatment is dialysis.

11. A device, comprising:
an electrode array having a first electrode, a second electrode, a third electrode and a fourth electrode that are axially aligned with each other, the electrodes being configured to be adhered to a patient, the first and fourth electrodes carrying two excitation signals at different frequencies and the second and third electrodes receiving a patient signal;
a base unit, configured to be connected to the electrode array, that generates the two excitation signals at the different frequencies and receives the patient signals; and
a fluid monitor computer system having a processor and a plurality of lines of instructions executed by the processor so that the processor is configured to:
receive the patient signals generated by the two different frequencies; and
determine a hydration of the patient during the treatment based on an extracellular impedance during the treatment.

12. The device of claim 11, wherein the processor is further configured to determine a ratio of the extracellular impedance at a start of the treatment to the extracellular impedance at a predetermined later time during the treatment and determine the hydration based on the determined ratio.

13. The device of claim 11, wherein a first frequency is 5 kHz and a second higher frequency is 100 kHz.

14. The method of claim 11, wherein the treatment is dialysis.

15. The device of claim 11, wherein the processor is further configured to determine one of a dry weight and euvolemia during the treatment based on an extracellular impedance during the treatment.

16. The device of claim 11, wherein the base unit is battery powered.

17. The device of claim 11, wherein the fluid monitor computer system has a display that is configured to display the determined hydration of the patent during the treatment.
